(19) United States
(12) Patent Application Publication
(10) Pub. No.: US 2016/0242944 A1
(43) Pub. Date: Aug. 25, 2016

(54) ORTHOPEDIC DEVICE FOR THE CORRECTION OF HALLUX VALGUS

(71) Applicant: Albert CIESIELSKI, Torun (PL)

(72) Inventor: Albert CIESIELSKI, Torun (PL)

(21) Appl. No.: 15/027,686

(22) PCT Filed: Oct. 7, 2013
(86) PCT No.: PCT/IB2013/059186

§ 371 Date: Apr. 6, 2016

(51) Int. Cl. A61F 5/01

(52) U.S. Cl. CPC A61F 5/019; A61F 5/0127; A61F 2005/0137

(57) ABSTRACT

The present invention relates to a device having a hinged splint made up of a shank of the big toe (1) and a shank of the metatarsus (2) which have an articulated connection. Both shanks (1, 2) are fitted with bands (11, 12) fastening the hinged splint to the patient's foot. The articulated connection of the sharks (1, 2) comprises the first (4) and the second (5) cylindrical hinge. The cylindrical hinges (4, 5) are connected by means of a central element (3) of the hinged splint, with the axes of rotation (6) of both cylindrical hinges (4, 5) being substantially parallel to each other.

### TECHNICAL FIELD

The invention concerns an orthopaedic device for the correction of incorrectly positioned big toes designed for people affected by deformities such as hallux valgus.

### BACKGROUND ART

Some simple separating devices are known and used for the treatment of deformities such as hallux valgus, having the form of a wedge inserted between the big toe and the second toe. In this way the big toe is pushed away from the incorrect position. One disadvantage of such devices is the fact that in order to apply the force needed to straighten the big toe out, the wedge supports itself against the neighbouring toes. As a result, a misalignment of the toes may occur.

A device for the treatment of hallux valgus is known from the German utility model DE1881215. In this solution, there is a splint running along the inner part of the foot. The device is fitted with a toe loop at the end of the big toe used to hold the toe. At the rear end the splint is so bowed that it can be positioned above the heel. As a result it possible to abduct the big toe and hold it in its normal position. One disadvantage of the device is that it is not comfortable in use and consequently patients are reluctant to wear it. In such case the therapeutic effect is insignificant.

A device for the correction of wrongly positioned toes is known from the U.S. Pat. No. 7,396,338, having a first fastening provision in the region of the big toe, a second fastening provision in the region of the central foot and a flexible splint which is held by the first and second fastening provisions and which is adapted to extend along an inner medial side of the foot. The flexible splint comprises two splint shanks and a hinge mechanism. The flexible splint is formed as a hinged flexible splint, articulated in a direction of flexion and extension of a toe. The hinge mechanism is adapted to be positioned on the main big toe joint. The flexible splint has a single pivot axis that corresponds approximately to the joint axis of the main big toe joint in the direction of flexion and extension. A disadvantage of the solution is the fact that the splint has only one pivot axis, which only approximately corresponds to the movements of the big toe in relation to the rest of the foot. The movements of the big toe when the device is worn are not completely natural and thus the therapeutic effect is only partial.

### DISCLOSURE OF INVENTION

The purpose of the invention is to develop an orthopaedic device for the correction of displacement of toes, which will be effective in the treatment of the hallux valgus condition. Furthermore, the device is also to be comfortable for the patients in use and suitable for wearing during everyday activities. It is also to correspond as much as possible to the natural movements of the big toe in relation to the foot during walking.

The device according to the invention is equipped with a hinged splint made up of a shank of the big toe and a shank of the metatarsus which have an articulated connection. Both shanks are fitted with bands fastening the hinged splint to the patient's foot. What distinguishes the device is that the articulation of the shanks comprises the first and the second cylindrical hinge. The cylindrical hinges are connected by means of a central element of the hinged splint. The axes of rotation of both cylindrical hinges are substantially parallel to each other.

In one embodiment of the device according to the invention, the first cylindrical hinge includes a round hole located at one end of the shank of the big toe and a pin fixed permanently to the central element of the hinged splint. The second cylindrical hinge includes a round hole located at one end of the shank of the metatarsus and a pin fixed permanently to the central element of the hinged splint. In another embodiment of the device according to the invention, the first cylindrical hinge is a round hole located at one end of the shank of the big toe and a pin fixed permanently to the central element of the hinged splint. The second cylindrical hinge includes a pin located at one end of the shank of the metatarsus and fixed permanently to the shank and a round hole in the central element of the hinged splint.

In another embodiment of the device according to the invention, the free ends of the pins of the cylindrical hinges have round snap-on caps. The outer diameters of the caps are greater than the diameter of the holes holding the pins on which the caps are positioned.

In another embodiment of the device according to the invention, the shank of the big toe and the shank of the metatarsus are close in shape to an oval with an axis of symmetry.

In another embodiment of the device according to the invention, the shank of the big toe and the shank of the metatarsus viewed in vertical section are concave in shape thus fitting in with the anatomic shape of the foot.

In another embodiment of the device according to the invention, the shank of the big toe and the shank of the metatarsus have longitudinal openings for the bands fastening the device to patient's foot.

In another embodiment of the device according to the invention, the shank of the big toe has two openings for the fastening band.

In another embodiment of the device according to the invention, the shank of the metatarsus has at least three openings for the band fastening the device to patient's foot.

In another embodiment of the device according to the invention, the shank of the big toe, the shank of the metatarsus and the central element of the hinged splint are made of plastic, advantageously of ABS (acrylonitrile butadiene styrene) or polycarbonate.

In another embodiment of the device according to the invention, the bands fastening the device to patient's foot are made of velour.

In another embodiment of the device according to the invention, the bands fastening the device to patient's foot are made of straps of material fitted with fasteners. Advantageously, the fastener is Velcro material. The fastening bands can go through the openings in the shanks.

In another embodiment of the device according to the invention, the central element of the splint is shaped like an axis symmetric figure.

In another embodiment of the device according to the invention, the axis of rotation of the first cylindrical hinge and the axis of rotation of the second cylindrical hinge are on the axis of symmetry of the central element of the splint.

In another embodiment of the device according to the invention, the central element of the splint is fitted with a cover whose outline corresponds to the outline of the central element. The cover covers the first and the second cylindrical hinge.

In another embodiment of the device according to the invention, the shank of the big toe has a wing on the lower edge extending towards the big toe.

In another embodiment of the device according to the invention, the end edges of the shank of the big toe and of the shank of the metatarsus with the holes for the pins of the cylindrical hinges are shaped like segments of circles concentric with the holes. These segments of the edges of the shanks have toothed sections interlocking and engaging with each other.

In another embodiment of the device according to the invention, the angle determining the lengths of the segments of circles providing the basis for the toothed sections of both the shanks is not greater than 180°.

In another embodiment of the device according to the invention, the radius of the segment of a circle constituting the edge of the end of the shank of the big toe with the toothed section is greater than the radius of the segment of a circle constituting the edge of the end of the shank of the metatarsus with the toothed section.

In another embodiment of the device according to the invention, both shanks are fitted with flat circular rings permanently fixed to the shanks with the axes of rotation of the cylindrical hinges going through the centre of the rings. The rings are connected in spots with each other on their circumferences with the use of a cord.

In another embodiment of the device according to the invention, the central element of the splint is fitted with a mechanism for changing the angle of the shank of the big toe in the horizontal plane.

In another embodiment of the device according to the invention, the mechanism for changing the angle of the shank of the big toe consists of an eccentric mechanism fitted with holes in which removable pins are positioned.

In another embodiment of the device according to the invention, the cylindrical hinges are fitted with devices blocking the vertical movement of the shanks. The device blocking the vertical movement of the shank comprises a number of holes in the shank being blocked, a single hole in the cover of the cylindrical hinges and a removable pin positioned in the said holes. The holes in the shank being blocked are located on the circumference of the circle through the centre of which runs the axis of rotation of the blocked shank.

In another embodiment of the device according to the invention, there is secured a separable elongated cushion on the surface of the shank of the metatarsus and the central element adjacent to the patient's foot. The cushion can be made up of two parts, and in such case one part is secured to the shank of the metatarsus and the second to the central element.

In another embodiment of the device according to the invention, the device is fitted with a pad fixed to the band fastening the shank of the metatarsus to the patient's foot.

The solution according to the invention makes it possible to obtain a device which owing to its small size and anatomical shape is comfortable for patients to use. Owing to the application of convexly shaped hinge mechanism, using the device, also in a shoe, during everyday activities causes no pain even when the patient has a bunion. Another advantage of the solution is the use of two axes of rotation. Owing to this solution, when the patient walks, the movement of the shank of the big toe in relation to the shank of the metatarsus corresponds to the movement of the bone of the big toe in relation to the metatarsal bone. As a result, the therapeutic effect is better than in the earlier solutions. At the same time, the use of two axes of rotation prevents unnatural bending of the big toe in relation to the rest of the foot.

### BRIEF DESCRIPTION OF DRAWINGS

Exemplary embodiments of the device according to the invention are presented on the drawings where

FIG. 1 depicts a side view of the first embodiment of the hinged splint, whereas

FIG. 2 shows a complete device on a bent foot.

FIG. 3 and

FIG. 4 show one of the variants of the first embodiment of the invention, intended for the left foot in front view and top view respectively, whereas

FIG. 5 presents another variant of the hinged splint in the same embodiment of the invention.

FIG. 6 shows the hinged splint in the second embodiment of the invention, where both shanks of the hinged splint are interlocked by means of the toothed sections.

FIG. 7 depicts the hinged splint in the third embodiment of the invention, where both shanks of the hinged splint are connected by means of a cord.

FIG. 8 shows a top view of the hinged splint in the fourth embodiment of the invention, where the device is fitted with a mechanism for changing the angle of the shanks, whereas

FIG. 9 shows the details of this mechanism in a side view.

FIG. 10 shows a side view the hinged splint in the fifth embodiment of the invention, where the device is fitted with devices blocking the movement of the shanks.

FIG. 11 shows a schematic presentation of the distribution of forces exerted on the foot when the device according to the invention is worn. Figures from 12 to 20 illustrate the hinged splint in the sixth embodiment of the invention, with

FIG. 12 showing a side view of the splint,

FIG. 13 showing a top view of the splint, and

FIG. 14 showing a cross section of the hinged splint along the vertical plane going through the axes of rotation of both shanks.

FIG. 15 shows an exploded view of the hinged splint in the sixth embodiment of the invention. Figures from 16 to 20 show individual components of the hinged splint in this embodiment of the invention in cross sections along the vertical plane as in FIG. 14, with

FIG. 16 depicting the cover fixing the shank of the big toe,

FIG. 17 depicting the cover fixing the shank of the metatarsus,

FIG. 18 depicting the central element of the splint,

FIG. 19 depicting the shank of the big toe, and

FIG. 20 depicting the shank of the metatarsus.

FIG. 21 shows a schematic top view presentation of the cushion separating the patient's foot and the splint of the device according to the invention, whereas

FIG. 22 and

FIG. 23 show a top view of two variants of the cushion.

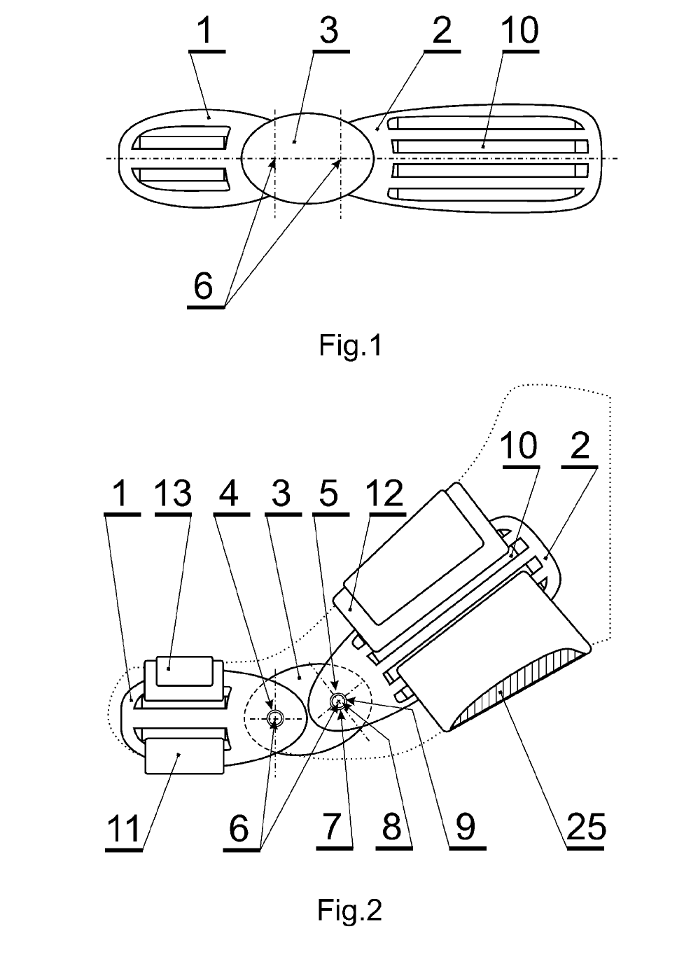

### MODE FOR CARRYING OUT INVENTION

In the first embodiment of the invention, the device has a hinged splint made up of the shank of the big toe 1, the shank of the metatarsus 2 and the intermediary element 3. The shank of the big toe 1 and the shank of the metatarsus 2 are oval in shape with a one axis of symmetry and in vertical section they are concave to fit in with the anatomic shape of the foot in areas where they are in contract with the foot. What's more, the size of the shank of the big toe 1 corresponds to the size of the big toe, while the shank of the metatarsus 2 is larger and extends from the metatarsophalangeal joint of the big toe towards the metatarsus. The shank of the big toe 1 is connected with the shank of the metatarsus 2 by means of the central element 3, which is elliptical in shape and is fitted from the side of the patient's foot. Moreover, in the vertical and horizontal section the central element 3 is convex in shape with the convexity extending outwards the foot. Owing to the convexity of the central element 3, the orthopaedic device can be used by patients with bunions without causing any additional pain. The central element 3 is connected with the shank of the big toe by means of the first cylindrical hinge 4, and with the shank of the metatarsus by means of the second cylindrical hinge 5.

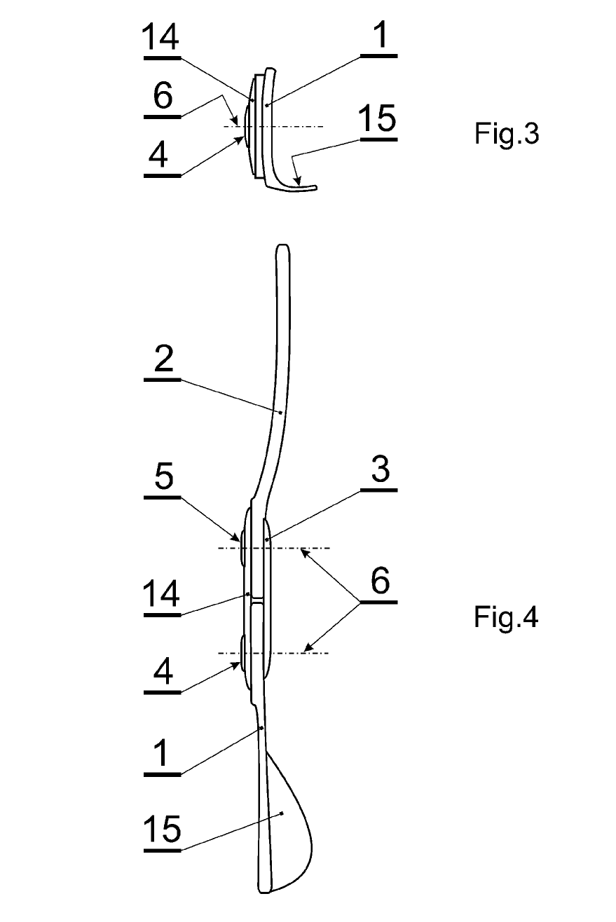

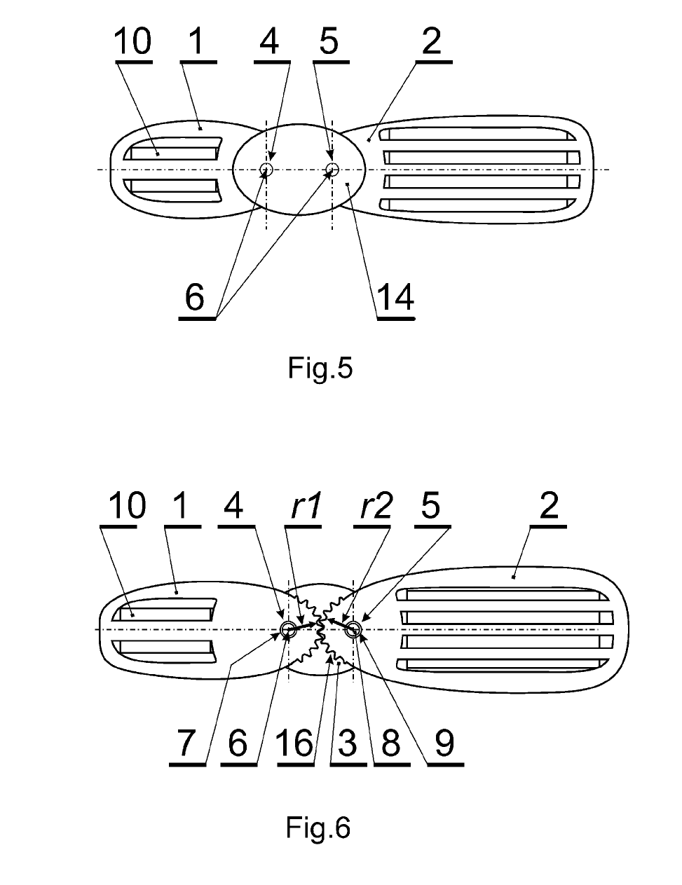

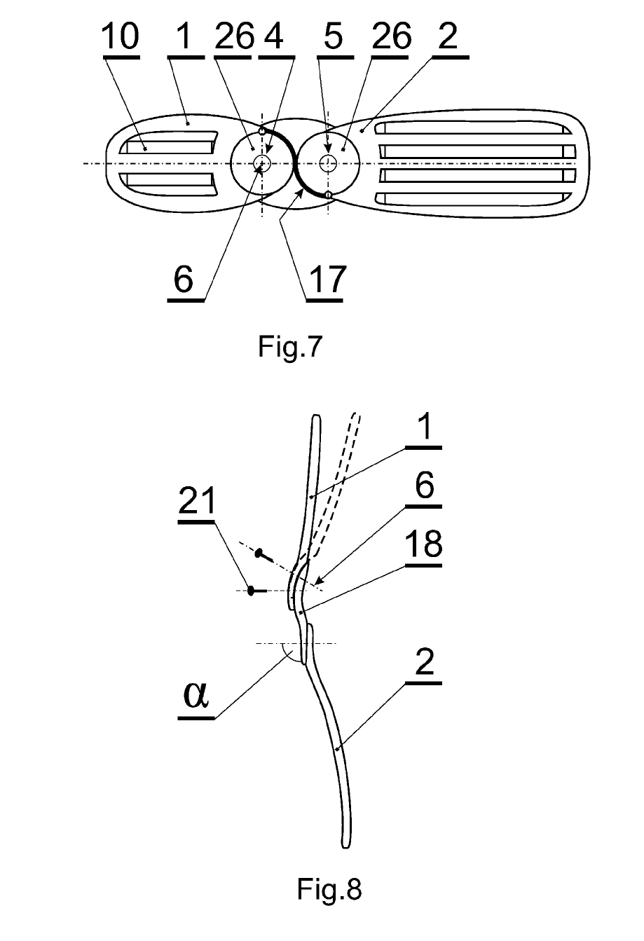

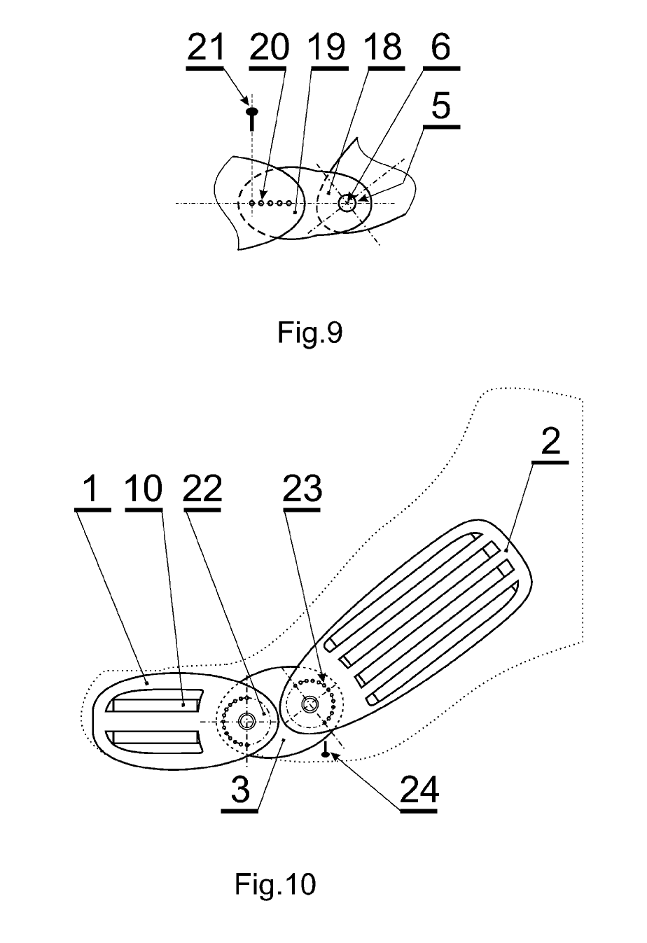

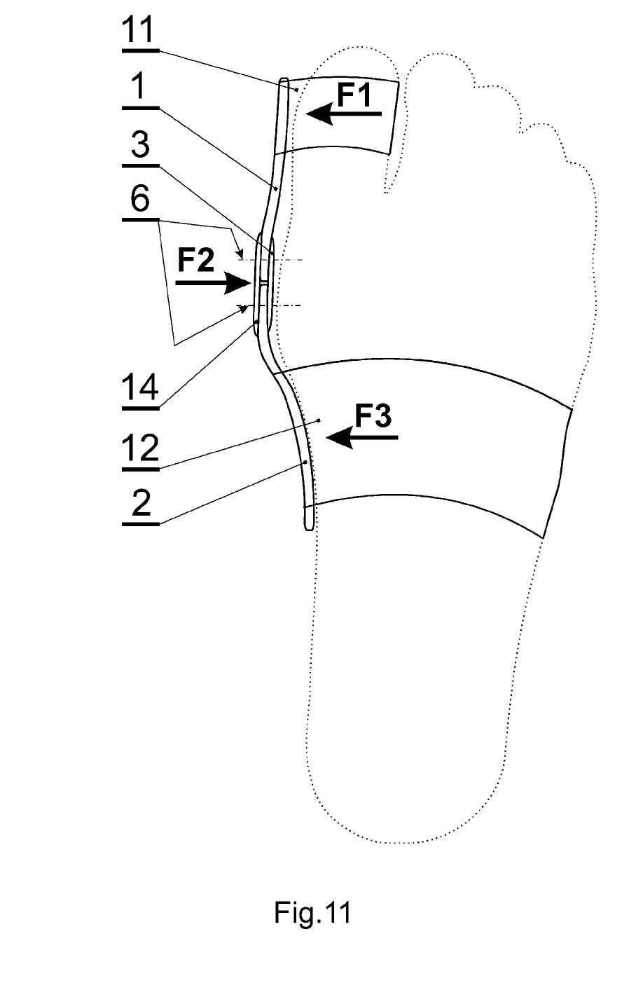

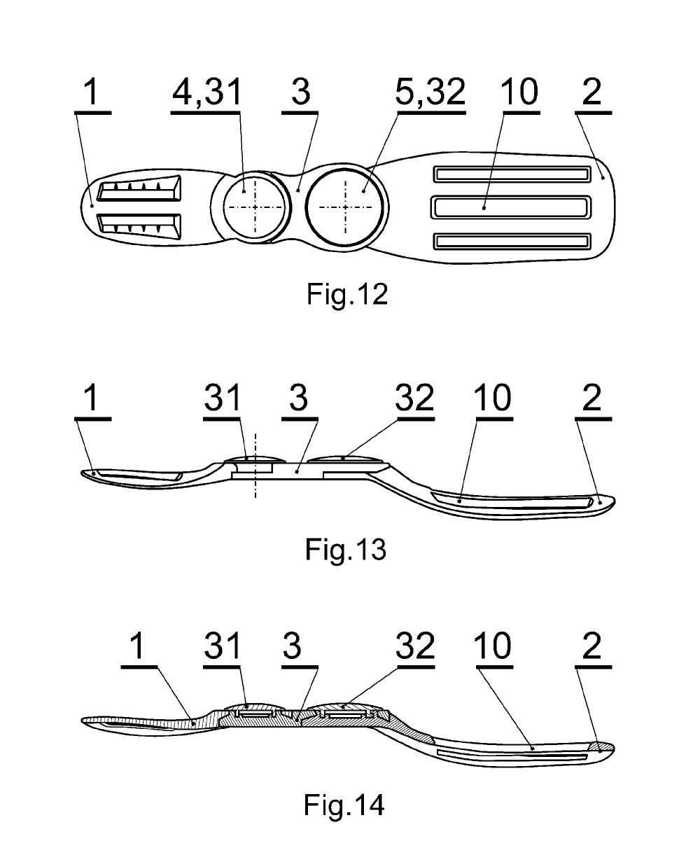

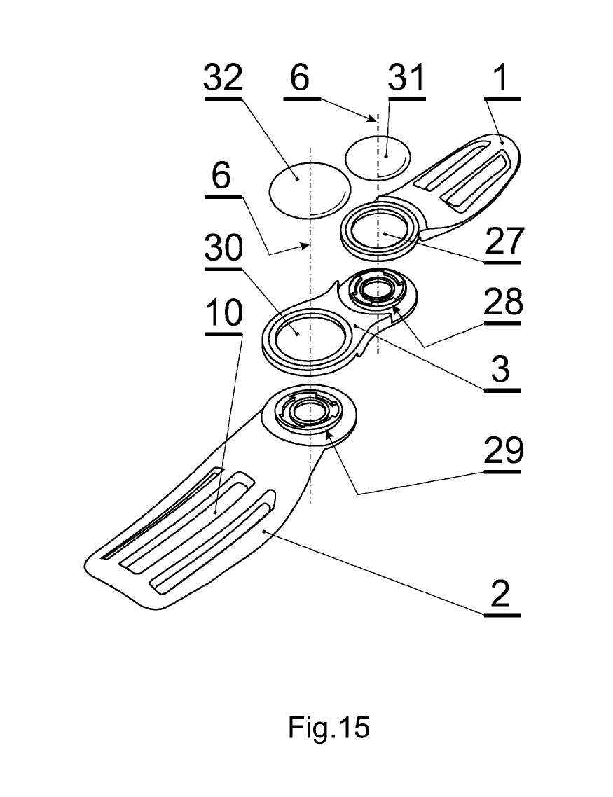

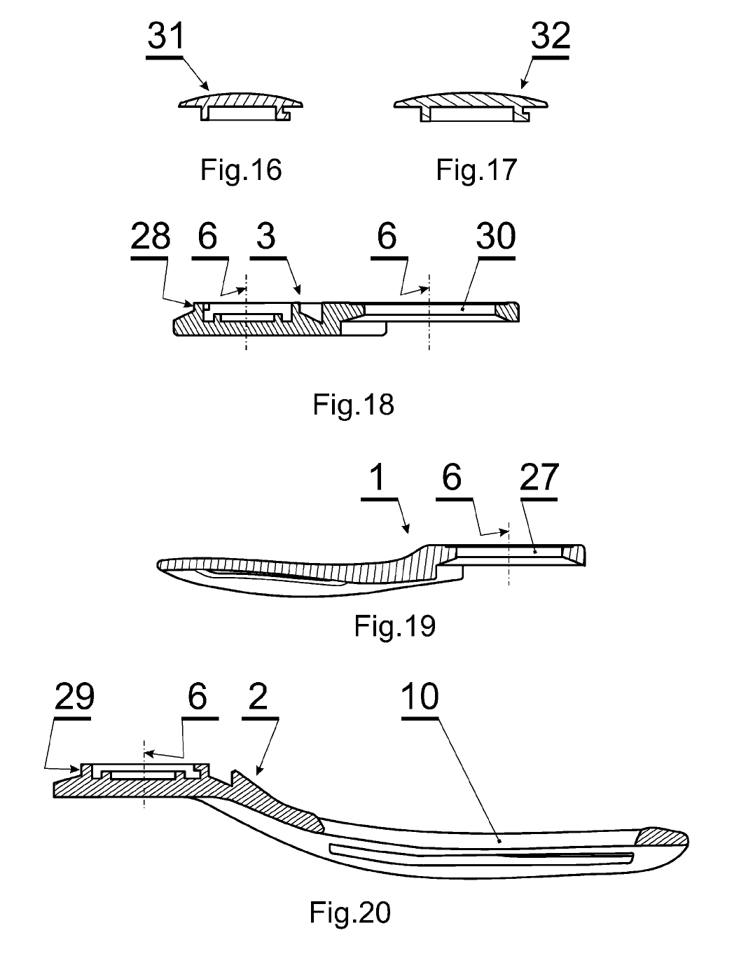

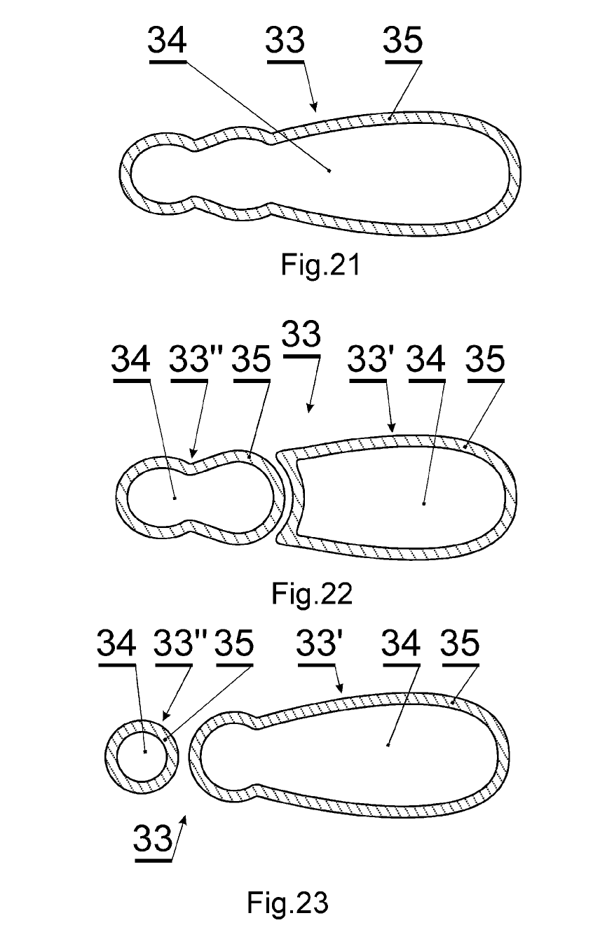

The axes of rotation 6 of the hinges 4 and 5 are positioned on the longer axis of symmetry of the ellipsis 3. The first cylindrical hinge 4 includes a round hole 7 located at one end of the shank of the big toe 1 and a pin 8 situated in the hole 7 and fixed permanently to the central element 3. The second cylindrical hinge 5 includes a round hole 7 located at one end of the shank of the metatarsus 2 and a pin 8 situated in this hole and fixed permanently to the central element 3. The pins 8 are prevented from falling out from the holes 7 in an uncontrolled manner by connectors 9. The shank of the big toe 1 has two longitudinal and mutually parallel openings 10, whereas the shank of the metatarsus 2 has four such longitudinal openings 10. The openings 10 on the shank of the big toe 1 are used for threading the fastening band 11 through them. The fastening band 11 holds the shank 1 at the big toe when the device is worn. The fastening band 11 abducts the big toe from the other toes with a force F1 by pulling it towards the shank of the big toe 1, thus correcting its wrong positioning. The openings 10 on the shank of the metatarsus 2 are meant for threading the fastening band 12 through them. The fastening band 12 holds the shank in a fixed position at the metatarsus. The fastening bands 11 and 12 are made of velour straps, and owing to the fact that they are connected with the hinged splint by being threaded through the openings 10 they can be easily disconnected for cleaning or replacement. The ends of the velour straps are fitted with fasteners 13 in the form of Velcro material providing for creation of the bands 11 and 12. In one variant of this embodiment, the central element 3 is fitted with an elliptical cap 14 covering the first cylindrical hinge 4 and the pin 8 of the second cylindrical hinge 5. Thanks to this, the moving shank of the big toe 1 and the moving, shank of the metatarsus 2 are protected at the connecting spot with the central element 3 against any undesired hooking up with shoe lining, sock, or foot skin. In another variant of this embodiment, shank of the big toe 1 has a wing 15 on the lower edge extending towards the big toe. The wing 15 is perpendicular to the shank of the big toe 1. The orthopaedic device shown in FIG. 3 and FIG. 4 is designed for use on a left foot. The wing 15 seen in a top view is shaped like an unsymmetrical arc, while in the vertical section it is concave in shape to better fit in with the shape of the big toe. The wing 15 provides for a better contact of the shank of the big toe 1 with the big toe. This reduces considerably the risk of the shank of the big toe 1 not cooperating with the big toe, especially at the time of bending. The orthopaedic device in this embodiment has a wing 15 extending towards the big toe of the left foot. The orthopaedic device is fitted with a pad 25 which is fixed by means of Velcro material to the fastening band 12 supporting the transverse arch of foot. The edges of the end of the shank of the big toe 1 and the shank of the metatarsus 2 with the holes 7 for the pins 8 are shaped like an arc. The hinged splint 1 is made of ABS (acrylonitrile butadiene styrene) or polycarbonate. When worn, the orthopaedic device exerts three forces on the foot: F1, F2 and F3. The vectors of the forces F1 and F3 go in the same direction, while the vector of the force F2 goes in the opposite direction. The force F1 is exerted on the big toe by the fastening band 11 fixed on the shank of the big toe 1 bringing the big toe out of the incorrect position. The force F3 is exerted by the fastening band 12 fixed on the shank of the metatarsus 2. The force F2 is exerted by the hinge mechanism 3 at the level of the metatarsophalangeal joint of the big toe.

In the second embodiment, the orthopaedic device differs from the first embodiment in that the edges of the ends of the shank of the big toe 1 and the shank of the metatarsus 2 with the holes 7 for the pins 8 are shaped like segments of circles which are concentric with the holes and have along those segments of the edges toothed sections 16 interlocking and engaging with each other. Owing to the toothed sections 16, the moving shank of the metatarsus 2 lifts or lowers the shank of the big toe 1. The angle determining the length of the segments of circles providing the basis for the toothed sections 16 is 150°, 75° on each side of the axis of symmetry of the shank 1 and 2. In one variant of this second embodiment, the radius r1 of the segment of a circle constituting the edge of the end of the shank of the big toe 1 and at the same time providing the basis for the toothed section 16 of the shank 1 can be twice as big as the radius r2 of the segment of a circle constituting the edge of the end of the shank of the metatarsus 2 and providing the basis for the toothed section 16 of the shank 2. Owing to this solution, the shank of the metatarsus 2 moves faster than the shank of the big toe 1.

A third embodiment of the invention differs from the first embodiment in that both shanks 1 and 2 are fitted with flat circular rings 26 permanently fixed to the shanks with the axes of rotation 6 of the cylindrical hinges 4 and 5 going through the centre of the rings. The rings 26 are connected in spots with each other on their circumferences with the use of a cord 17. Owing to this solution, the movement of the shank of the big toe 1 and the shank of the metatarsus 2 is correlated, just as is the case with the solution with toothed sections 16.

In a fourth embodiment, the orthopaedic device described in the first embodiment is fitted with a mechanism 18 for changing the angle. The mechanism 18 for changing the angle includes an eccentric mechanism 19 fitted with holes 20 in which pins 21 are positioned. Owing to the mechanism 18 for changing the angle, it is possible to change the angle α in the horizontal plane between the shank of the big toe 1 and the first axis of rotation 6 by ±30° in comparison to the default position, which in the embodiment is 90°.

In a fifth embodiment, the cylindrical hinges 4 and 5 are fitted with devices 22 blocking the vertical movement of the shanks 1 and 2. The device 22 blocking the vertical movement of the shank 1 or 2 comprises a number of holes 23 in the shank being blocked, a single analogical hole in the cover 14 not shown in FIG. 10 and a removable pin 24 positioned in the holes 23. The holes are located on the circumference of the circle through the centre of which runs the axis of rotation 6 of the blocked shank 1 or 2. The position in which the shanks 1 or 2 are blocked depends on the choice of the hole 23 into which the removable pin 24 is inserted.

A sixth embodiment of the invention differs from the first embodiment in the method of connection of the components of the hinged splint and the number of openings 10, three in this case, in the shank of the metatarsus 2. The first cylindrical hinge 4 is a round hole 27 located at the end of the shank of the big toe 1 which is free from the longitudinal openings 10 and a pin 28 fixed permanently to the central element 3. The second cylindrical hinge 5 is a pin 29 located at the end of the shank of the metatarsus 2 which is free from the longitudinal openings 10 and permanently fixed to the shank 2 and a round hole 30 in the central element 3. On the free ends of the pins 28 and 29 snap-on caps 31 and 32 are fixed. The outer diameters of the caps 31 and 32 are greater than the diameters of the holes 27 and 30 holding the pins 28 and 29. The caps 31 and 32 prevent the cylindrical hinges 4 and 5 from disconnecting in an uncontrolled manner. The diameter of the holes 27 and 30 and consequently the diameter of the pins 28 and 29 is greater in comparison to the diameters in the previous embodiments, which advantageously affects the strength of the cylindrical hinges 4 and 5.

In each of the embodiments described above, an elongated cushion 33 can be positioned inside the caving in the shank of the metatarsus 2 and at the level of the central element 3. The cushion 33 has the form of a gel insert 34 enclosed on both sides with layers of velour fabric permanently connected with each other along the circumference 35 by means of pressure welding. The cushion 33 can be fixed to the splint of the device by means of Velcro material. The cushion 33 protects the skin on patient's foot against a direct contact with the material of the shank of the metatarsus 2 and the central element 3, and also reduces the accumulation of the force exerted by the device at the level of a bunion on the metatarsophalangeal joint of the big toe. In order to facilitate the operation of the second cylindrical hinge, the cushion 33 can be made up of two parts 33′ and 33″, and in such case one part 33′ is secured to the shank of the metatarsus 2 and the other part 33″ to the central element 3. The cushion 33 can also be made of other soft materials such as polyurethane, polyester, silicone, technogel, polymer gel, latex, caoutchouc, rubber, synthetic leather, natural leather or neoprene.

1. An orthopaedic device for the correction of hallux valgus fitted with a hinged splint made up of a shank of the big toe and a shank of the metatarsus which have an articulated connection, both equipped with bands fastening the hinged splint to patient's foot, wherein the articulated connection of the shanks (1, 2) is composed of the first cylindrical hinge (4) and the second cylindrical hinge (5), the hinges (4, 5) are connected by means of the central element (3) of the hinged splint, with the axes of rotation (6) of both cylindrical hinges (4, 5) being substantially parallel to each other.

2. The device according to claim 1, wherein the first cylindrical hinge (4) is a round hole (7) located at one end of the shank of the big toe (1) and a pin (8) fixed permanently to the central element (3), while the second cylindrical hinge (5) is a round hole (7) located at one end of the shank of the metatarsus (2) and a pin (8) fixed permanently to the central element (3).

3. The device according to claim 1, wherein the first cylindrical hinge (4) is a round hole (27) located at one end of the shank of the big toe (1) and a pin (28) fixed permanently to the central element (3), while the second cylindrical hinge (5) includes a pin (29) located at one end of the shank of the metatarsus (2) and fixed permanently to the shank (2) and a round hole (30) in the central element (3).

4. The device according to claim 3, wherein the free ends of the pins (28, 29) have round snap-on caps (31, 32) whose outer diameters are greater than the diameter of the holes (27, 30) holding the pins (28, 29) on which the caps are positioned.

5. The device according to claim 1, wherein the shank of the big toe (1) and the shank of the metatarsus (2) are close in shape to an oval with an axis of symmetry.

6. The device according to claim 1, wherein the shank of the big toe (1) and the shank of the metatarsus (2) viewed in vertical section are concave in shape thus fitting in with the anatomic shape of the foot.

7. The device according to claim 1, wherein the shank of the big toe (1) and the shank of the metatarsus (2) have longitudinal openings (10) for the fastening bands (11, 12).

8. The device according to claim 7, wherein the shank of the big toe (1) has two openings (10) for the fastening band (11).

9. The device according to claim 7, wherein the shank of the metatarsus (2) has at least three openings (10) for the fastening band (12).

10. The device according to claim 1, wherein the shank of the big toe (1), the shank of the metatarsus (2) and the central element (3) are made of plastic.

11. The device according to claim 10, wherein the plastic is ABS (acrylonitrile butadiene styrene) or polycarbonate.

12. The device according to claim 1, wherein the fastening bands (11, 12) are made of velour material.

13. The device according to claim 1, wherein the fastening bands (11) and (12) are straps of material fitted with fasteners (13), advantageously in the form of Velcro material and go through the openings (10) in the shanks (1, 2).

14. (canceled)

15. The device according to claim 1, wherein the central element (3) is shaped like an axis symmetric figure.

16. The device according to claim 15, wherein the axis of rotation of the first cylindrical hinge (4) and axis of rotation of the second cylindrical hinge (5) are on the axis of symmetry of the central element (3).

17. The device according to claim 1, wherein the central element (3) is fitted with a cover (14) whose outline corresponds to the outline of the central element (3) and which covers the first (4) and the second (5) cylindrical hinge.

18. The device according to claim 1, wherein the shank of the big toe (1) has a wing (15) on its lower edge extending towards the big toe.

19. The device according to claim 2, wherein the end edges of the shank of the big toe (1) and of the shank of the metatarsus (2) with the holes (7) for the pins (8) are shaped like segments of circles concentric with the holes (7) and these segments of the edges of the shanks (1, 2) have toothed sections (16) interlocking and engaging with each other.

20. The device according to claim 19, wherein the angle determining the lengths of the segments of circles providing the basis for the toothed sections (16) is not greater than 180°.

21. The device according to claim 19, wherein the radius (r1) of the segment of a circle constituting the edge of the end of the shank of the big toe (1) is greater than the radius (r2) of the segment of a circle constituting the edge of the end of the shank of the metatarsus (2).

22. The device according to claim 2, wherein the shanks (1, 2) are fitted with flat circular rings (26) permanently fixed to the shanks (1, 2) with the axes of rotation (6) of the cylindrical hinges (4, 5) going through the centre of the rings (26) and wherein the rings (26) are connected in spots with each other on their circumferences with the use of a cord (17).

23. The device according to claim 2, wherein the central element (3) is fitted with a mechanism (18) for changing the angle of the shank of the big toe (1) in the horizontal plane.

24. The device according to claim 23, wherein the mechanism (18) for changing the angle consists of an eccentric mechanism (19) fitted with holes (20) in which removable pins (21) are positioned.

25. The device according to claim 17, wherein the cylindrical hinges (4, 5) are fitted with devices (22) blocking the vertical movement of the shanks (1, 2), such device (22) blocking the vertical movement of the shank (1, 2) comprises a number of holes (23) in the shank being blocked (1, 2), a single hole (23) in the cover (14) and a removable pin (24) positioned in the holes (23) while the holes (23) in the shank (1, 2) being blocked are located on the circumference of the circle through the centre of which runs the axis of rotation (6) of the blocked shank (1, 2).

26. The device according to claim 1, wherein on the surfaces of the shank of the metatarsus (2) and the central element (3) adjacent to the patient's foot there is secured a separable elongated cushion (33).

27. The device according to claim 26, wherein the cushion (33) is made up of two parts and one part (33′) of the cushion (33) is secured to the shank of the metatarsus (2) while the other part (33″) of the cushion (33) is secured to the central element (3).

28. The device according to claim 1, wherein the device is fitted with a pad (25) fixed to the band (12) fastening the shank of the metatarsus (2).
